(19) United States
(12) Patent Application Publication
(10) Pub. No.: US 2008/0038349 A1
(43) Pub. Date: Feb. 14, 2008

(54) Peptide-Pna Chimera Targeting Inducible Nitric Oxide Synthetase

(76) Inventor: Lamensdorf Itschak, Modiin (IL)

Correspondence Address: PEARL COHEN ZEDEK LATZER, LLP, 1500 BROADWAY 12TH FLOOR, NEW YORK, NY, 10036, US

(21) Appl. No.: 10/589,755

(22) PCT Filed: Feb. 10, 2005
(86) PCT No.: PCT/IL05/00170

§ 371 Date: Jun. 4, 2007

Related U.S. Application Data
(60) Provisional application No. 60/544,353, filed on Feb. 17, 2004.

(51) Int. Cl. A61K 38/00; A61K 9/14; A61K 9/28; A61P 37/00; C07K 2/00; C07K 7/06; C07K 7/08; C12N 15/09

(52) U.S. Cl. 424/474; 424/489; 435/455; 514/2; 530/300; 530/326; 530/329

(57) ABSTRACT

Peptide Nucleic Acid (PNA) antisense specific for inducible nitric oxide (iNOS) wherein the PNA compound is composed from nucleic acid sequence targeting iNOS, a peptide ligand which binds to a specific receptor, a positively charge moiety and an hydrophobic moiety. The invention also relates to the use of these compounds for the treatment of multiple sclerosis, neurodegenerative disorders, acute brain insults or other diseases where iNOS is involved.

### FIELD OF THE INVENTION

The invention provides compounds comprising a peptide nucleic acid (PNA) antisense specific for inducible nitric oxide (iNOS), a peptide ligand which binds to a specific receptor and a positively charge peptide moiety with lysosomatic properties, useful in the delivery of a PNA antisense across a cellular membrane including the blood brain barrier. The invention also relates to the use of these compounds for the treatment of multiple sclerosis, neurodegenerative disorders, acute brain insults or other diseases where iNOS is involved.

### BACKGROUND OF THE INVENTION

Nitric oxide (NO) is a mediator involved in signaling in the cardiovascular, gastrointestinal, genitourinary, respiratory and nervous systems, and disordered NO generation has been implicated in a wide range of diseases.

In inflammatory, infectious, ischemic, and neurodegenerative pathologies of the central nervous system (CNS) glia become “activated” by inflammatory mediators, and express new proteins such as the inducible isoform of nitric oxide synthase (iNOS). Nitric oxide (NO), synthesized by iNOS appears to be a key mediator in glial-induced neuronal death. The high sensitivity of neurons to NO is partly due to NO causing inhibition of respiration, rapid glutamate release from both astrocytes and neurons, and subsequent excitotoxic death of the neurons.

Neurodegeneratives diseases such as Parlinson's (PD), Alzheimer's (AD), Huntington's (HD) and amyotrophic lateral sclerosis (ALS) have been related to mitochondrial dysfunction, production of reactive oxygen species, and accompanying oxidative stress. The applications of selective iNOS inhibitors against neurodegenerative diseases have potential as a therapeutic agent in treating this ailment.

Several studies on Alzheimer's disease (AD) reveled that reactive microglia associated with the β-amyloid plaques initiate a sequence of inflammatory events integral to the AD process. Studies have shown that amyloid peptides activate a tyrosine kinase-based signaling response in mouse microglia and human monocitic cell line resulting in the production of neurotoxic secretory products, proinflammatory cytokines and reactive oxygen species.

The production of NO and subsequent generation of peroxynitrite from iNOS activation is involved in neuronal damage through an apoptotic process. In addition, it has been found that the expression of iNOS in astrocytes is induced via NF-κβ-dependent mechanism. Inhibition of iNOS has shown a significant reduction of the associated toxicity, ergo the diminution of the cell damage rate has been observed. The use of non-steroidal anti-inflammatory drugs (NSAIDs) showed that the down regulation of cytokine levels and iNOS expression reduced cell death.

Inhibition of the complex I of the mitochondrial respiratory chain induces Parkinson's disease (PD) in primates and humans. Latest studies have shown the relationship between iNOS and a remarkable increase in damage in PD mice model, intoxicated with 1-methyl-4-phenyl-1,2,3,6-tetrahydropyridine (MPTP). This model proposed that dopaminergic fibers are destroyed by the toxic effect of MPTP and iNOS.

MPTP and nNOS showed a toxic effect that is not focused on the entire destruction of all dopamine fibres and its metabolite. MPTP toxicity is related with the NO produced by nNOS causing a primary damage to the dopaminergic fibers. This effect is involved in the activation of iNOS, which will produce NO in charge of attacking essentially neuronal cell bodies located in the substantia negra pars compacta. The utilization of selective iNOS and nNOS inhibitors might have a potential application to slow down or even stop the development of PD.

Huntington's disease (HD) is an inherited disease that causes severe neurodegenerative and neuroinflammatory disturbances. The genetic defect in Huntington's Disease (HD) has been recognized as an abnormal expanded trinucleotide (CAG) repeated in a gene located on the short arm of chromosome 4. Over activation of the glutamate receptors associated with excitotoxic processes results in a massive release of NO.

Experimental models where mitochondrial damage and HD are related have been done, suggesting the excitotoxicity to be an important factor in this pathogenesis. The inhibition of complex II of the mitochondrial respiratory chain induces HD in primates and humans. The HD models used have shown evidence that glial cells in the CNS can produce NO in vivo in response to stimulation by cytokines and that this production is mediated by iNOS. Other models based in N-methyl-D aspartate (NMDA) excitotoxicity have shown to be susceptible to inhibitors of iNOS.

Several studies suggested that selective nitric oxide-mediated oxidative damage in the motor system plays a part in the pathomechanism of the neuronal degeneration in the spinal cord of sporadic amyotrophic lateral sclerosis (ALS). Degenerating spinal motor neurons in familial and sporadic ALS are typically surrounded by reactive astrocytes expressing the inducible form of NO synthase (iNOS), suggesting that astroglia may have a pathogenic role in ALS in a peroxynitrite-dependent mechanisms.

Encephalitis is a brain inflammation of viral or other microbial origin. Viral encephalitis has shown to be related with the upregulation of NO and cytokines in animal models of Venezuelan equine encephalitis and Murray Valley encephalitis.

iNOS isoform is involved in the regulation of the blood brain barrier (BBB), through the generation of NO and its posterior conversion to peroxinitrite. It have been suggested that NO has a double role in viral encephalitis. The first role is to assist the immune protection against the unrelenting infection of the virus and the second one is its contribution in the resulting pathology. The therapeutic application of iNOS inhibitors against encephalitides can be used as an additional target in the battle against these diseases, including HIV-1 encephalitis, which is one of the most studied diseases in the last decades.

Multiple sclerosis (MS) is a disease that can affect any area of the brain and spinal cord. Experimental autoimmune encephalomyelitis (EAE) is a model of multiple sclerosis (MS). This experimental central nervous system (CNS) disease can be induced in rats by immunization with components of the CNS myelin sheath. The iNOS isoform has been implicated in the pathogenesis of multiple sclerosis. In a rodent model of experimental autoimmune encephalitis (EAE), the expression of iNOS in the CNS correlates with the severity of the disease. Recently, researchers have been found the presence of activated macrophages, expressing high levels of iNOS and evidence of peroxynitrite formation in brain tissue from patients and animal models with MS. Also the presence of neopterin a precursor of a cofactor in iNOS activation has been observed in the cerebrospinal fluid of MS patients. Increased levels of NO and iNOS mRNA have been detected in the CNS of animals with EAE. The application of inhibitors of iNOS has inhibited the disease. Significant benefits of NOS inhibition by single inhibitors or a mixture of drugs have also been reported in EAE. The study of iNOS functionality in MS, has shown to be of vital importance in the development of approaches to treat this disease.

Circumstantial evidence suggests that NO has a role in several features of the disease, including disruption of the blood-brain barrier, oligodendrocyte injury and demyelination, axonal degeneration, and that it contributes to the loss of function by impairment of axonal conduction. Several studies indicated that inhibition of iNOS activity should diminish disease progress.

Corticosteroids, which are being used in the treatment of MS, inhibit iNOS. The main mechanism of corticosteroid activity on iNOS is reduction in protein synthesis, not-destabilization (3). It was also been suggested that the therapeutic effect of Interferon-beta-1-b in MS may be partly due to suppression of pathogenic NO production.

Antisense drugs are complementary strands of small segments of DNA, or DNA analogues, that are linked together in short chains (called oligonucleotides; ODN). This short chain is designed to bind to a specific sequence of nucleotides in its mRNA target. By binding to the mRNA target responsible for the generation of disease causing proteins, antisense drugs are able to prevent production of the protein encoded by the target mRNA. Thus, this technology provides a highly specific strategy for targeting a wide range of diseases at the genetic level.

The use of antisense targeting iNOS for therapeutic applications was studied in several models of CNS disorders. Inhibition of iNOS expression in spinal cord injury model (SCI) was found to be beneficial in reducing several pathophysiological processes (significantly retard neuronal cell death rostral and caudal to the injury site) after SCI. Furthermore, it was demonstrated that the antisense inhibition of iNOS was more efficacious than currently available pharmacological agents.

Antisense oligodeoxynucleotide (ODN) directed against iNOS reduced the lesion volume and significantly improved recovery of sensorimotor functions in a model of transient focal cerebral ischemia in rats providing new evidence that inhibition of iNOS may be of interest for the treatment of stroke.

Peptide Nucleic Acid (PNA) is third generational nucleic acid chemistry, which is now being used in conjunction with antisense technology. PNA chemistry is unique in that the sugar and phosphate linkages are replaced by neutral and biologically more stable peptide linkages. PNAs are hydrophilic macromolecules and their administration required disruption of plasma membrane. Therefore, unmodified/naked PNA molecules pass poorly through the cell membrane and do not have useful therapeutic applications.

Serving as a filter to the brain, the Blood Brain Barrier is a highly complex endothelial interface that separates the brain from the blood compartment. It blocks the delivery of 98% of small-molecule drugs, and nearly all large molecule drugs to the brain. Both ODNs and PNAs cannot cross the endothelial cellular membrane of the blood brain barrier (BBB) effectively, thus, preventing the possible use of these technologies in developing drugs for CNS disorders.

It is known in the art that peptides can cross the blood brain barrier via receptor mediated transcytosis. This process involved binding of the receptor and peptide at one side of the BBB (e.g. the luminal membrane), translocation of the receptor-peptide complex through the cytoplasm, and dissociation of the peptide from the receptor on the external surface of the abluminal membrane.

It have been shown that monobiotinylated peptide nucleic acid, conjugated to transferrin receptor monoclonal antibody the OX26/SA vector undergoes a 28-fold increase in BBB transport following intravenous injection of the conjugate. Moreover, PNAs retain affinity for target RNA despite conjugation to the BBB drug targeting system. However, this technology is inefficient, complex and has a potential for immunogenic activation. Furthermore, this approach will unlikely be active in vivo without an endosomal release function built into it.

It is known in the art that peptide containing lysine, cysteine and histidine can transfect cells in culture with a better efficiency than peptides without histidine. The polycationic peptide CHK6HC was found to improved transfection of cells in culture.

### SUMMARY OF THE INVENTION

In one embodiment, the invention provides a molecule represented by any one of the formulas I-IV:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein

N is a nucleic acid sequence targeting iNOS, L is a peptide ligand, which binds to a specific receptor and P is a positively charge moiety; and H is an hydrophobic moiety herein r is an integer of 1-25, t is an integer of 1-25, s is an integer of 1-25, q is an integer of 0-20, and x is an integer of 1-20.

In another embodiment, the present invention provides a molecule represented by any one of the formulas I-IV:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein N is a nucleic acid sequence in a length of 1-100 bases, L is a peptide ligand which binds to a specific receptor, P is a positively charge moiety; and H is an hydrophobic moiety

wherein r is an integer of 1-25, t is an integer of 1-25, s is an integer of 1-25, q is an integer of 0-20, and x is an integer of 1-20.

In another embodiment of the invention, there is provided a molecule represented by any one of the formulas V-VIII:

[(L)t-(PNA)r-(H)q-(P)s]x

[(L)t-(H)q-(PNA)r-(P)s]x

[(P)s-(PNA)r-(H)q-(L)t]x

[(P)s-(H)q-(PNA)r-(L)t]x

wherein the PNA is PNA sequence targeting iNOS DNA, cDNA, RNA or mRNA and is in a length of 1-100 bases, L is a peptide ligand which binds to a specific receptor and P is a positively charge moiety; and wherein r is an integer of 1-25, t is an integer of 1-25, s is an integer of 0-25, q is an integer of 0-20 and x is an integer of 1-20.

In another embodiment, the present invention provides a molecule represented by any one of the formulas V-VIII:

[(L)t-(PNA)r-(H)q-(P)s]x

[(L)t-(H)q-(PNA)r-(P)s]x

[(P)s-(PNA)r-(H)q-(L)t]x

[(P)s-(H)q-(PNA)r-(L)t]x

wherein the PNA sequence is a PNA sequence targeting iNOS mRNA or DNA, wherein the length of said PNA sequence is 1-20 bases, L is a peptide ligand which binds to a specific receptor and P is a positively charge moiety; and wherein r is an integer of 1-25, t is an integer of 1-25, s is an integer of 0-25, q is an integer of 0-20 and x is an integer of 1-20.

In one embodiment, the present invention further provides a method for delivering a molecule across a cellular membrane and to the brain across the blood brain barrier.

In another embodiment, there is provided a method for intracellular targeting of a nucleic acid sequence targeting iNOS DNA, cDNA, RNA or mRNA, to an intracellular organelle comprising the step of applying to a cell an effective amount of one or more molecules of the invention.

In another embodiment, there is provided a method for delivering a nucleic acid sequence targeting iNOS DNA, cDNA, RNA or mRNA to the brain across the blood brain barrier, said method comprising the step of administering to a subject a composition or a molecules according to the invention.

In another embodiment, the invention provides a method for the treatment, prevention and control of a disease resulted in inhibition of iNOS translation, said method comprising administering to a subject an effective amount of one or more molecules, or composition comprising the same, according to the embodiments of the invention.

In another embodiment, the invention provides a method for the treatment, prevention and control of multiple sclerosis, said method comprising administering to a subject an effective amount of one or more molecules, or composition comprising the same, according to the embodiments of the invention.

### BRIEF DESCRIPTION OF THE DRAWINGS

The present invention will be understood and appreciated more fully from the following detailed description taken in conjunction with the appended drawings in which:

FIG. 1 demonstrates hybridization properties of conjugated PNA.

FIG. 2 (a) and FIG. 2 (b) demonstrate the effect of conjugated PNA targeting iNOS mRNA on (a) mean clinical score and (b) disease burden in multiple sclerosis mice model (b).

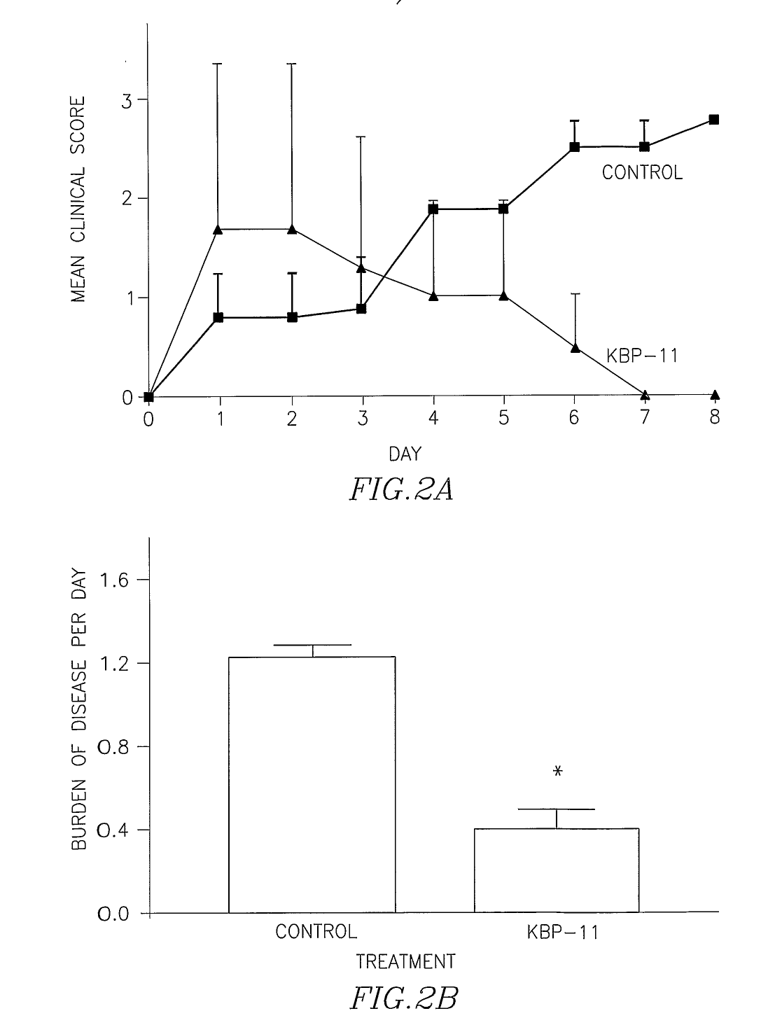

FIG. 3 demonstrates immunohistochemical staining of iNOS protein in cortical sample of conjugated-PNA treated or control non-treated animals.

### DETAILED DESCRIPTION OF THE PRESENT INVENTION

In one embodiment, the invention provides a molecule represented by any one of the formulas I-IV:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein

N is a nucleic acid sequence targeting iNOS bases, L is a peptide ligand which binds to a specific receptor, P is a positively charge moiety; and H is a hydrophobic moiety and wherein r is an integer of 1-25, t is an integer of 1-25, s is an integer of 1-25, q is an integer of 0-20, and x is an integer of 1-20.

In one embodiment of the present invention, N is a nucleic acid sequence targeting iNOS in a length of 1-25 bases. In another embodiment, N is a nucleic acid sequence in a length of 1-10 bases. In another embodiment, N is a nucleic acid sequence in a length of 1-20 bases. In another embodiment, N is a nucleic acid sequence in a length of 10-20 bases. In another embodiment, N is a nucleic acid sequence in a length of 20-30 bases. In another embodiment, N is a nucleic acid sequence in a length of 30-40 bases. In another embodiment, N is a nucleic acid sequence in a length of 40-50 bases. In another embodiment, N is a nucleic acid sequence in a length of 50-100 bases.

In another embodiment, the present invention provides a molecule represented by any one of the formulas I-IV:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein N is a nucleic acid sequence in a length of 1-100 bases, L is a peptide ligand which binds to a specific receptor, P is a positively charge moiety; and H is an hydrophobic moiety and wherein r is an integer of 1-25, t is an integer of 1-25, s is an integer of 1-25, q is an integer of 0-20, and x is an integer of 1-20.

In another embodiment, there is provided a molecule represented by any one of the formulas V-VIII:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein the PNA is PNA sequence targeting iNOS DNA, cDNA, RNA or mRNA, L is a peptide ligand which binds to a specific receptor and P is a positively charge moiety; and wherein r is an integer of 1-25, t is an integer of 1-25 s is an integer of 0-25, q is an integer of 0-20 and x is an integer of 1-20.

In another embodiment, the invention provides a molecule represented by any one of the formulas V-VIII:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein the PNA is PNA sequence targeting iNOS DNA, cDNA, RNA or mRNA and is 1-100 bases, L is a peptide ligand which binds to a specific receptor and P is a positively charge moiety; and wherein r is an integer of 1-25, t is an integer of 1-25 s is an integer of 0-25, q is an integer of 0-20 and x is an integer of 1-20.

In one embodiment, the present invention provides a molecule represented by any one of the formulas I-IV:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein N is a nucleic acid sequence in a length of 1-25 bases, L is a peptide ligand which binds to a specific receptor and P is a positively charge moiety; an wherein r is an integer of 1-25, t is an integer of 125 s is an integer of 1-25, q is an integer of 0-20, and x is an integer of 1-20.

In one embodiment, the present invention provides a molecule represented by any one of the formulas V-VIII:

[(L)t-(N)r-(H)q-(P)s]x

[(L)t-(H)q-(N)r-(P)s]x

[(P)s-(N)r-(H)q-(L)t]x

[(P)s-(H)q-(N)r-(L)t]x

wherein the length of said PNA sequence is 5-25 bases targeting iNOS (accession number M84373), mRNA or DNA, L is a peptide ligand which binds to a specific receptor and P is a positively charge moiety; and wherein r is an integer of 1-25, t is an integer of 1-25 is an integer of 0-25, q is an integer of 0-20 and x is an integer of 1-20.

Peptide Nucleic Acid (PNA) is third generational nucleic acid chemistry, which is now being used in conjunction with antisense technology. PNA chemistry is unique in that the sugar and phosphate linkages are replaced by neutral and biologically more stable peptide linkages. PNAs are hydrophilic macromolecules and their administration required disruption of plasma membrane. Therefore, unmodified/naked PNA molecules pass poorly through the cell membrane and do not have useful therapeutic applications. In order to improve their cellular uptake, PNAs were conjugated to delivery moieties such as positively charged peptide, receptor ligands or hydrophobic moiety. In vitro, PNAs targeting macrophags iNOS (3′-Gly-CTTTCTCCTTTTCC-Lys 5′) specifically diminished its expression.

PNA can be used in another embodiment of the invention as a spacer between the peptide ligand and positively charged moiety. This chimera can cross the blood brain barrier either via receptor-mediated transcytosis (RMT) or absorptive mediated transcytosis (AMT). Thus, the PNA/nucleic acid is functioning as a spacer diminishing steric/electrostatic interaction between the two peptide moiety. In another embodiment, PNA/nucleic acid may be used as an antisense moiety, an antigene moiety or a gene modulator moiety.

In one embodiment of the present invention, the length of the PNA sequence targeting iNOS is 1-100 bases. In another embodiment, the length of the PNA sequence is 1-10 bases. In another embodiment, the length of the PNA sequence is 1-20 bases. In another embodiment, the length of the PNA sequence is 10-20 bases. In another embodiment, the length of the PNA sequence is 20-30 bases. In another embodiment, the length of the PNA sequence is 30-40 bases. In another embodiment, the length of the PNA sequence is 40-50 bases. In another embodiment, the length of the PNA sequence is 50-100 bases.

In one embodiment of the present invention, q is an integer of 0-20. In another embodiment, q is an integer of 2-10. In another embodiment, q is an integer of 6-16. In another embodiment, q is an integer of 7-11. In another embodiment, q is 8. In another embodiment, q is 9. In another embodiment, q is 0.

In one embodiment of the present invention, r is an integer of 0-20. In another embodiment, r is an integer of 1-10. In another embodiment, r is an integer of 10-20. In another embodiment, r is an integer of 5-10. In another embodiment, r is an integer of 11-15. In another embodiment, r is an integer of 2-5.

In one embodiment of the present invention, s is an integer of 0-25. In another embodiment, s is an integer of 2-15. In another embodiment, s is an integer of 2-6. In another embodiment, s is an integer of 6-10. In another embodiment, s is 4. In another embodiment, s is 0.

In one embodiment of the present invention, t is an integer of 1-25. In one embodiment of the present invention, s is an integer of 20-25. In another embodiment, s is an integer of 10-15. In another embodiment, s is an integer of 2-6. In another embodiment, s is an integer of 6-10. In another embodiment, s is 4. In another embodiment, s is 0.

In one embodiment of the present invention, x is an integer of 1-20. In another embodiment, x is an integer of 2-15. In another embodiment, s is an integer of5-10.

In one embodiment of the present invention, the nucleic acid sequence is iNOS RNA, mRNA. In another embodiment the nucleic acid sequence is iNOS cDNA. In another embodiment the nucleic acid sequence is a DNA. In another embodiment the nucleic acid sequence is a DNA analog. In another embodiment of the invention the nucleic acid sequence is a PNA. In another embodiment the nucleic acid sequence is a PNA morpholino. In another embodiment the nucleic acid sequence is an aminoethylprolyl (aep) PNA. In another embodiment of the invention, the nucleic acid sequence is a pyrrolidinyl PNA. In another embodiment of the invention, the nucleic acid sequence is an oligonucleotide. In another embodiment the nucleic acid sequence is an oligonucleotide analog. In another embodiment the nucleic acid sequence is a ribozyme. In another embodiment the nucleic acid sequence is an RNAi. In another embodiment, the nucleic acid sequence is modified PNA (aepPNA).

In one embodiment of the present invention, the nucleic acid sequence is an antisense. In another embodiment, the nucleic acid sequence is an antigene. In another embodiment, the nucleic acid sequence is a decoy function. In one embodiment of the invention, the nucleic acid sequence has a neutral charge. In another embodiment of the invention, the nucleic acid sequence is negatively charged. In one embodiment of the present invention, the nucleic acid sequence is positively charged. In one embodiment of the present invention, nucleic acid sequence is in antisense orientation to an endogenous sequence.

The term “nucleotide” refers in the invention to a subunit of DNA or RNA consisting of a nitrogenous base (adenine, guanine, thymine, or cytosine in DNA; adenine, guanine, uracil, or cytosine in RNA), a phosphate molecule, and a sugar molecule (deoxyribose in DNA and ribose in RNA). Thousands of nucleotides are linked to form a DNA or RNA molecule. The term nucleotide further describes protected guanine; pseudo-guanine or protected pseudo-guanine (2,6-diaminopurine); protected adenine; protected cytosine; pseudo-cytosine or protected pseudo-cytosine, pseudo-isocytosine or protected pseudo-isocytosine; protected uracil.

The term “oligonucleotide” refers in the invention to a molecule usually composed of 25 or fewer nucleotides.

The term “antisense” refers in the invention to a nucleic acid sequence that has a sequence exactly opposite to an mRNA molecule made by the body; binds to the mRNA molecule to prevent a protein from being made.

The term “peptide nucleic acids” (PNAs) refers to molecules that in certain respects are similar to oligonucleotide analogs however in other very important respects their structure is very different. In peptide nucleic acids, the deoxyribose phosphate backbone of oligonucleotides has been replaced with a backbone more akin to a peptide than a sugar phosphodiester. Each subunit has a naturally occurring or non-naturally occurring base attached to this backbone. A non-limiting example is a backbone constructed of repeating units of N-(2-aminoethyl)glycine or analogues thereof having a nucleobase attached thereto via a linker such as a carboxymethyl moiety or analogues thereof to the nitrogen atom of the glycine portion of the unit. The units are coupled together via amide bonds formed between the carboxyl group of the glycine moiety and the amine group of the aminoethyl moiety. The nucleobase can be one of the four common nucleobases of nucleic acids or they can include other natural or synthetic nucleobases. Because of the radical deviation from the deoxyribose backbone, these molecules were named peptide nucleic acids.

The term “antigene” refers to molecules, which bind to double-stranded DNA. Antigenes can in one embodiment enhance gene expression. In another embodiment, antigenes can inhibit gene expression in cells.

In one embodiment, the t invention provides an improved method for the delivery of PNA-based nucleic acid whereas the PNA is used as spacer between the peptide ligand and the positively charged moiety. The chimera can cross the blood brain barrier either via RMT or AMT. The PNA/nucleic acid is functioning as a spacer diminishing steric/electrostatic interaction between the two peptide moiety. In another embodiment of the invention, the PNA/nucleic acid is used as an antisense moiety, targeting iNOS RNA, mRNA or DNA. In another embodiment, the PNA/nucleic acid molecule is used as an apolar peptide-like moiety in an amphiphlic brain vector.

In one embodiment, the invention provides an improved method for delivery of PNA-based nucleic acid targeting iNOS's RNA, mRNA or DNA through the BBB following addition of hydrophobic moiety to the PNA-peptide chimera. Said compound can cross the BBB as a result of its ampiphilic structure, as a result of receptor mediated transcytosis or absorptive mediated transcytosis

In one embodiment of the present invention, the peptide ligand binds a receptor to transferrin. In another embodiment, the peptide ligand binds a receptor to insulin. In another embodiment, the peptide ligand binds a receptor to insulin growth factor. In another embodiment, the insulin growth factor is an insulin growth factor-I. In another embodiment, the insulin growth factor is an insulin growth factor-II. In another embodiment, the peptide ligand binds a receptor to leptin. In another embodiment, the peptide ligand that binds a receptor is HAIYPRH. In another embodiment, the peptide ligand that binds a receptor is THRPPMWSPVWP.

In one embodiment of the present invention, the hydrophobic moiety is a nucleic acid. In another embodiment, the hydrophobic moiety is a nucleic acid analog. In another embodiment, the hydrophobic moiety is a hydrophobic peptide. In another embodiment of the invention, the hydrophobic moiety is a lipid acid. In another embodiment of the invention, the hydrophobic moiety is a lipid molecules. In another embodiment of the invention, the hydrophobic moiety is octanol. In another embodiment the hydrophobic moiety is cholesterol. In another embodiment the hydrophobic moiety is a hydrophobic peptide protecting group. In another embodiment the hydrophobic moiety is adamantine. In another embodiment the hydrophobic moiety is pyrene. In another embodiment the hydrophobic moiety is eicosenoic acid. In another embodiment the hydrophobic moiety is a C(6-16) glyceride lipid. In another embodiment the hydrophobic moiety is phenoxazine. In another embodiment the hydrophobic moiety is a DMT group. In another embodiment the hydrophobic moiety is cholenic acid. In another embodiment the hydrophobic moiety is lithocholic acid. In another embodiment the hydrophobic moiety is myristic acid. In another embodiment the hydrophobic moiety is palmitic acid. In another embodiment the hydrophobic moiety is a heptadecyl group. In another embodiment the hydrophobic moiety is hexadecylglycerol. In another embodiment the hydrophobic moiety is a geranyloxyhexyl group. In another embodiment the hydrophobic moiety is hexadecylamine. In another embodiment the hydrophobic moiety is dihydrotestosterone. In another embodiment the hydrophobic moiety is 1-pyrene butyric acid. In another embodiment the hydrophobic moiety is alkanoic acid. In another embodiment the hydrophobic moiety is alkanol. In another embodiment the hydrophobic moiety is and any derivatives of the above mentioned moieties. In one embodiment of the present invention, the alkanoic acid is represented by the structure R-(CH₂)n-COOH, wherein n is an integer of 1-20 and R is a linear or branched alkyl. In another embodiment n is an integer of 6-16. In one embodiment of the present invention, the alkanol is represented by the structure R-(CH₂)n-OH, wherein n an integer of 1-20 and R is a linear or branched alkyl. In another embodiment n is an integer of 6-16. In one embodiment of the present invention, the lipid acid is undecanoic acid. In another embodiment, the lipid acid is docosahexanenonic acid. In one embodiment of the invention, the hydrophobic peptide protecting group is Fmoc. In another embodiment of the invention, the hydrophobic peptide protecting group is Tboc.

As contemplated herein, an “alkyl” group refers to a saturated aliphatic hydrocarbon, including straight-chain, branched-chain and cyclic alkyl groups. In one embodiment, the alkyl group has 1-4 carbons. In another embodiment, the alkyl group is a methyl group. In another embodiment, the alkyl group is an ethyl group. In another embodiment, the alkyl group is a propyl group. In another embodiment, the alkyl group is a butyl group. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

In one embodiment of the present invention, the molecule is designed to have a positively charged moieties conjugated to the N or C terminals of the modified-PNA in accordance with the invention.

In one embodiment of the present invention, the positively charge moiety is a nucleic acid sequence. In another embodiment, the positively charge moiety is a nucleic acid analog. In another embodiment, the positively charge moiety is a PNA. In another embodiment, the positively charge moiety is a positively charged peptide. In another embodiment, the positively charge moiety is a peptidomimetic. In another embodiment, the positively charge moiety is a polycations. In another embodiment, the positively charge moiety is a histidine. In another embodiment, the positively charge moiety is an imidazole group. In another embodiment, the positively charge moiety is 2-O-aminopropyl. In another embodiment, the positively charge moiety is 2-O-dimethylaminopropyl. In another embodiment, the positively charge moiety is 2-O-imidazolyl-ethyl. In another embodiment, the positively charge moiety is 2-O-aminoethylamino-oxyethyl. In another embodiment, the positively charge moiety is 2-dimethylaminoethyl-oxyethyl. In another embodiment, the positively charge moiety is and any derivative of the above mentioned moieties. In another embodiment, the positively charge moiety is arginine. In another embodiment, the positively charge moiety is D-arginine. In another embodiment, the positively charge moiety is polyarginine. In another embodiment, the positively charge moiety is polyamine. In another embodiment, the positively charge moiety is guanidine. In another embodiment, the polyamine is spermine. In another embodiment, the polyamine is spermidine. In another embodiment, the polyamine is putricine.

In one embodiment of the present invention, the positively charge moiety is a cationic peptide. In another embodiment, the cationic peptide is CHK6HC.

In one embodiment of the present invention, the positively charge moiety is CK4HK3C. In another embodiment, the positively charge moiety is CHK6HC. In another embodiment, the positively charge moiety is CHK3HK2HC. In another embodiment, the positively charge moiety is C(HK)4C. In another embodiment, the positively charge moiety is CHKHKHHKHC.

In one embodiment of the present invention, the PNA sequence is CTT TCT CCT TTT CC. In another embodiment, the PNA is an (aminoethylprolyl (aep) PNA)1-20.

In one embodiment of the present invention, the PNA sequence is CTT TCT CCT TTT CC. In another embodiment, the PNA is a (Guanidine-Based Peptide Nucleic Acids (GPNA) 1-20.

In one embodiment of the present invention, the peptide ligand, the hydrophobic moiety, the PNA/nucleic acid sequence and the positively charge moiety are linked to each other directly via peptide bonds.

In one embodiment, the present invention provides a molecule comprising CHK6HC-(PNA)r-HAIYPRH. In one embodiment, the present invention provides a molecule comprising CHK6HC-(PNA)r-THRPPMWSPVWP. In one embodiment, the present invention provides a molecule comprising CK4HK3C-(PNA)r-HAIYPRH. In one embodiment, the present invention provides a molecule comprising CK4HK3C-(PNA)r -THRPPMWSPVWP. In one embodiment, the present invention provides a molecule comprising CHK3HK2HC-(PNA)r-HAIYPRH. In one embodiment, the present invention provides a molecule comprising CHK3HK2HC-(PNA)r -THRPPMWSPVWP. In one embodiment, the present invention provides a molecule comprising C(HK)4C-(PNA)r-HAIYPRH. In one embodiment, the present invention provides a molecule comprising C(HK)4C-(PNA)r -THRPPMWSPVWP. In one embodiment, the present invention provides a molecule comprising CHKHKHHKHC-(PNA)r-HAIYPRH. In one embodiment, the present invention provides a molecule comprising CHKHKHHKHC-(PNA)r-THRPPMWSPVWP. In one embodiment of the present invention r is 5-25. In another embodiment r is 5-10. In another embodiment r is 10-20. In another embodiment r is 10-15.

In one embodiment, the present invention provides a molecule comprising-HAIYPRH-(PNA)r-CHK6HC. In one embodiment, the present invention provides a molecule comprising THRPPMWSPVWP-(PNA)r -CHK6HC. In one embodiment, the present invention provides a molecule comprising HAIYPRH-(PNA)r-CK4HK3C. In one embodiment, the present invention provides a molecule comprising THRPPMWSPVWP-(PNA)r -CK4HK3C.

In one embodiment, the present invention provides a molecule comprising HAIYPRH-(PNA)r-CHK3HKHC. In one embodiment, the present invention provides a molecule comprising THRPPMWSPVWP-(PNA)r -CHK3HK2HC. In one embodiment, the present invention provides a molecule comprising HAIYPRH-(PNA)r-C(HK)4C. In one embodiment, the present invention provides a molecule comprising THRPPMWSPVWP-(PNA)r -C(HK)4C. In one embodiment, the present invention provides a molecule comprising HAIYPRH-(PNA)r-CHKHKHHHKHC. In one embodiment, the present invention provides a molecule comprising THRPPMWSPVWP-(PNA)r -CHKHKHHKHC. In one embodiment of the present invention r is 5-25. In another embodiment r is 5-10. In another embodiment r is 10-20. In another embodiment r is 10-15.

In one embodiment, the present invention provides a molecule comprising CHK6HC-CTT TCT CCT TTT CC-THRPPMWSPVWP. In one embodiment, the present invention provides a molecule comprising CHK6HC -CTT TCT CCT TTT CC -HAIYPRH. In one embodiment, the present invention provides a molecule comprising THRPPMWSPVWP-CTT TCT CCT TTT CC -CHK6HC. In one embodiment, the present invention provides a molecule comprising HAIYPRH-CTT TCT CCT TTT CC-CHK6HC. In one embodiment, the present invention provides a molecule comprising CTT TCT CCT TTT CC-THRPPMWSPVWP.

In one embodiment, the present invention provides a molecule comprising docosahexanenonic acid (DHA)-PNA(1-20 bases)-(Arg)4. In one embodiment, the present invention provides a molecule comprising undecanoic acid-PNA (1-20 bases)-(Arg)4. In one embodiment, the present invention provides a molecule comprising PNA-DHA-(Arg)4. In one embodiment, the present invention provides a molecule comprising undecanoic acid-CTT TCT CCT TTT CC-(Arg)2-15. In one embodiment, the present invention provides a molecule comprising docosahexanenonic acid-CTT TCT CCT TTT CC-(Arg)2-15. In one embodiment, the present invention provides a molecule comprising undecanoic acid-CTT TCT CCT TTT CC-(Arg)2-15.

In one embodiment, the present invention provides a molecule comprising DHA-PNA(0-20 bases)-(deoxynucleic guanidine-DNG)1-20. In one embodiment, the present invention provides a molecule comprising PNA(0-20 bases)-DHA-(DNG)1-20. In one embodiment, the present invention provides a molecule comprising DHA-PNA(0-20 bases)-(aminoethylprolyl (aep) PNA)1-20. In one embodiment, the present invention provides a molecule comprising PNA(0-20 bases)-DHA-(aepPNA)1-20. In one embodiment, the present invention provides a molecule comprising Guanidine-Based Peptide Nucleic Acids (GPNA) 1-20 -PNA(0-20 bases)-(GPNA)1-20 ; (Pyrrolidinyl PNA)1-20 -PNA(0-20 bases)-(aepPNA)1-20. In one embodiment, the present invention provides a molecule comprising PNA(0-20 bases)-(Pyrrolidinyl PNA)1-20 -(aepPNA)1-20. In one embodiment of the present invention, the PNA is in a length of 5-10 bases. In another embodiment, the PNA is in a length of 1-5 bases. In another embodiment, the PNA is in a length of 10-15 bases.

In one embodiment, the present invention provides a molecule comprising CTT TCT CCT TTT CC-Phe-Leu-Phe-Leu-Arg-Arg. In one embodiment, the present invention provides a molecule comprising (C)8-CTT TCT CCT TTT CC-(D-Arg)6. In one embodiment, the present invention provides a molecule comprising PEG -(C)8-TTT GCT CTT ACT CAT-(D-Arg)6. In one embodiment, the present invention provides a molecule comprising PEG-CTT TCT CCT TTT CC-(C)8-(D-Arg)6. In one embodiment, the present invention provides a molecule comprising TTT GCT CTT ACT CAT -PEG -(C)8-(D-Arg)6. In one embodiment, the present invention provides a molecule comprising (Arg)2 CTT TCT CCT TTT CC-Phe-Leu-Phe-Leu-Phe-Leu. In one embodiment, the present invention provides a molecule comprising (Arg)6-CTT TCT CCT TTT CC -Phe-Leu-Phe-Leu-Phe-Leu. In one embodiment, the present invention provides a molecule comprising CTT TCT CCT TTT CC-(Phe-Lhe)2-3-(Arg)2-6. In one embodiment, the present invention provides a molecule comprising (Phe-Lhe)2-3-TTT GCT CTT ACT CAT-(Arg)2-6. In one embodiment, the present invention provides a molecule comprising Cys-His-(Lys)6His-Cys-PNA(1-20 bases)-(Phe-Lhe)2-3. In one embodiment, the present invention provides a molecule comprising Cys-His-(Lys)6His-Cys-Cys PNA(1-20 bases)-(Phe-Lhe)2-3. In one embodiment, the present invention provides a molecule comprising (CH2)9-CO-(C CTT TCT CCT TTT CC)-Gly-Arg-Arg-Arg-Lys. In one embodiment of the present invention, the PNA is in a length of 5-10 bases.

In one embodiment, the present invention provides a molecule comprising DHA-PNA (1-20 bases)-putricine. In one embodiment, the present invention provides a molecule comprising PNA (1-20 bases)-DHA-putricine. In one embodiment, the present invention provides a molecule comprising undecanoic acid-PNA(1-20 bases)-Putricine. In one embodiment, the present invention provides a molecule comprising PNA(1-20 bases)-undecanoic acid -putricine. In one embodiment, the present invention provides a molecule comprising undecanoic-PNA(1-20 bases)-spermine. In one embodiment, the present invention provides a molecule comprising PNA(1-20 bases)-undecanoic acid-spermine. In one embodiment, the present invention provides a molecule comprising undecanoic acid-PNA(1-20 bases)-spermidine. In one embodiment, the present invention provides a molecule comprising PNA(1-20 bases)-undecanoic acid-spermidine. In one embodiment of the present invention, the PNA is in a length of 5-10 bases.

In one embodiment, the present invention provides a molecule comprising Fmoc-PNA(1-20 bases)-putricine. In one embodiment, the present invention provides a molecule comprising. TBoc-PNA(1-20 bases)-putricine. In one embodiment, the present invention provides a molecule comprising docosahexanenonic acid-PNA(1-20 bases)-Diethylenetriamine. In one embodiment, the present invention provides a molecule comprising docosahexanenonic acid-PNA(1-20 bases)-polyethylenimine. In one embodiment, the present invention provides a molecule comprising allyl substituted PNA(1-20 bases)-polyethylenimine. In one embodiment, the present invention provides a molecule comprising chloro and/or bromo-halogenated PNA(1-20 bases)-polyethylenimine. In one embodiment, the present invention provides a molecule comprising allyl substituted PNA(1-20 bases)-(Arg/D-Arg)2-10. In one embodiment, the present invention provides a molecule comprising chloro and/or bromo-halogenated substituted PNA(1-20 bases)-(Arg/D-Arg)2-10. In one embodiment of the present invention, the PNA is in a length of 5-10 bases. In one embodiment of the present invention, the number of Arg/D-Arg groups is 2-6. In another embodiment, the number of Arg/D-Arg groups is 4.

In one embodiment of the invention, the molecule further comprising a linker moiety linking between the peptide ligand, the hydrophobic moiety, the PNA/nucleic acid sequence and the positively charge moiety. In another embodiment, the linker moiety is polyethylene glycol (PEG). In another embodiment, the molecular weight of said PEG is in the range of 2000-40,000. In another embodiment, the linker moiety is a disulfide. In another embodiment, the linker moiety is an amide. In another embodiment, the linker moiety is an amine. In another embodiment, the linker moiety is an oxyamine. In another embodiment, the linker moiety is an oxyimine. In another embodiment, the linker moiety is a morpholine. In another embodiment, the linker moiety is a thioether. In another embodiment, the linker moiety is thiourea sulfonamide. In another embodiment, the linker moiety is an ether. In another embodiment, the linker moiety is an ester. In another embodiment, the linker moiety is a carbonate. In another embodiment, the linker moiety is a carbamate. In another embodiment, the linker moiety is guanidine. In another embodiment, the linker moiety is avidin. In another embodiment, the linker moiety is strepavidin. In another embodiment, the linker moiety is biotin. In another embodiment, the linker moiety is praline. In another embodiment, the linker moiety is lysine. In another embodiment, the linker moiety is cysteine.

In one embodiment of the present invention, the liable linker or peptide bond to polyethylene glycol conjugated to the molecule improves phramacokinetic properties and overcomes possible side effects induced by the amphiphilic PNA. In one embodiment of the present invention, the linker is conjugated to the molecule via a known technology.

In one embodiment, the present invention provides a molecule comprising Cys-His-(Lys)₆His-Cys-Cys-PNA(1-20 bases)-(Phe-Lhe)₂₋₃. In one embodiment, the present invention provides a molecule comprising Cys-His-(Lys)6His-Cys-Cys-PNA(1-20 bases)-avidin/strepavidin-biotin-(Phe-Lhe)₂₋₃. In one embodiment, the present invention provides a molecule comprising Cys-His-(Lys)₆His-Cys-Cys-PNA(1-20 bases)-(Phe-Lhe)₂₋₃. In one embodiment, the present invention provides a molecule comprising Cys-His-(Lys)₆His-Cys-Cys-PNA(1-20 bases)-(Phe-Lhe)₂₋₃. In one embodiment, the present invention provides a molecule comprising (Arg/D-Arg)₂₋₆-Proline -Lys-CTT TCT CCT TTT CC-Cys-Cys-(Phe-Lhe)₂₋₃. In one embodiment, the present invention provides a molecule comprising (Arg/D-Arg)₂₋₆-CGC TGG GC-Cys-Cys -(Phe-Lhe)₂₋₃. In one embodiment, the present invention provides a molecule comprising (Arg)₂₋₆-PEG-CTT TCT CCT TTT CC-Cys-Cys-(Phe-Lhe)₂₋₃. In one embodiment, the present invention provides a molecule comprising (Arg/D-Arg)₂₋₆-PEG-Cys-Cys-CTT TCT CCT TTT CC-Cys-Cys -(Phe-Lhe)₂₋₆. In one embodiment of the present invention, the PNA is in a length of 5-10 bases. In one embodiment of the present invention, the number of Arg/D-Arg groups is 2-6. In another embodiment, the number of Arg/D-Arg groups is 4. In one embodiment, the present invention the molecular weight of said polyethylene glycol is in the range of 2000-40,000.

In one embodiment, the present invention further provides a composition comprising as an active ingredient an effective amount of one or more molecules according to claim 1, together with one or more pharmaceutically acceptable excipients or adjuvant. In one embodiment of the present invention, the composition is formulated for oral or parenteral administration. In another embodiment, the composition is formulated as uncoated tablets, coated tablets, pills, capsules, powder or suspension. In another embodiment, the composition is formulated for intravenous administration. In another embodiment, the composition is formulated intranasal administration. In another embodiment, the composition is formulated administration via aerosols. In another embodiment, the composition is formulated for transdermal administration. In another embodiment, the composition is formulated in an ointment, cream or gel form. In another embodiment, the compositions of the present invention are formulated in a liquid dosage form. Examples of suitable liquid dosage forms include solutions or suspensions in water, pharmaceutically acceptable fats and oils, alcohols or other organic solvents, including esters, emulsions, syrups or elixirs, solutions and/or suspensions.

Suitable excipients and carriers can be solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, and coloring agents, flavoring agents, flow-inducing agents, and melting agents. Liquid dosage forms may contain, for example, suitable solvents, preservatives, emulsifying agents, suspending agents, diluents, sweeteners, thickeners, and melting agents Parenteral and intravenous forms should also include minerals and other materials to make them compatible with the type of injection or delivery system chosen.

In one embodiment, the present invention further provides a method for delivering a PNA/nucleic acid sequence targeting iNOS RNA, DNA or mRNA across a cellular membrane comprising the step of applying to a cell an effective amount of one or more molecules according to the invention. In one embodiment of the present invention, the cell is an endothelial cell. In another embodiment, the cell is a neuronal cell. In another embodiment, the cell is a glial cell. In another embodiment, the cell is an oligodendrocyte cell. In another embodiment, the cell is a muscle cell.

In one embodiment, the present invention provides a method for the improved delivery of PNAs targeting iNOS RNA, DNA or mRNA into mammalian cells. In one embodiment, the present invention provides an amphiphilic PNA chimeric moiety with improved neuronal, endothelial delivery properties.

In one embodiment, the present invention further provides a method for intracellular targeting of a PNA/nucleic acid sequence targeting iNOS RNA, DNA or mRNA to an intracellular organelle comprising the step of applying to a cell an effective amount of one or more molecules according to the invention. In one embodiment of the present invention, charge distribution, length of the apolar PNA/nucleic acid chain targeting iNOS RNA, DNA or mRNA and hydrophobicity can affect sub-cellular compartization. In one embodiment, the present invention provides a method for intracellular targeting of a PNA/nucleic acid sequence to an intracellular organelle comprising the step of applying to a cell an effective amount of one or more molecules of the invention, wherein the molecules cross the nuclear membrane.

In one embodiment, the present invention further provides a method for delivering a PNA/nucleic acid sequence targeting iNOS RNA, DNA or mRNA in the brain, which crosses the blood brain barrier, comprising the step of administering to a subject an effective amount of one or more molecules according to the embodiments of the invention. In another embodiment, the present invention provides a method for delivering a PNA/nucleic acid sequence targeting iNOS RNA, DNA or mRNA to the brain, which crosses the blood brain barrier, comprising the step of administering to a subject a composition according to the embodiments of the invention.

In one embodiment of the invention, polyarginine oligomers are used to improve BBB penetration of PNA-based constructs targeting iNOS RNA, DNA or mRNA. In another embodiment, arginine guanido groups are used to improve BBB penetration of PNA-based constructs. In one embodiment of the present invention, the increased brain uptake through the BBB is via guanidine basic amino acid transporters. In another embodiment, the increased brain uptake is the result of augmented AMT as a result of increased permeability surface due to the presence of positive charge.

In one embodiment of the present invention, polyamines are used to improve BBB penetration of PNA-based constructs targeting iNOS RNA, DNA or mRNA. In another embodiment, polyamine is putrescine. In another embodiment, polyamine is sperimidine. In another embodiment, polyamine is putrescine. In another embodiment, polyamine is spermine. In one embodiment of the present invention, the increased brain uptake through the BBB is via polyamine transporters. In another embodiment, the increased brain uptake is the result of augmented AMT as a result of increased permeability surface due to the presence of positive charge.

In one embodiment, the present invention further provides a method for modulating iNOS gene expression, said method said method comprising administering to a subject an effective amount of one or more molecules according to the invention. In another embodiment, the present invention provides a method for modulating gene expression; said method said method comprising administering to a subject one a composition according to the invention.

In one embodiment, the present invention provides a kit comprising an effective amount of one or more molecules according to the invention. In another embodiment, the kit allows gene labeling. In another embodiment, the kit further comprising labeling and/or reaction buffers. In another embodiment, the molecule is conjugated to a fluorescent label, a colorimetric label, a radiolabel label or a chemical label.

In one embodiment, the present invention further provides a method for the treatment, prevention and control of multiple sclerosis, said method comprising administering to a subject an effective amount of one or more molecules according to the invention. In another embodiment, the present invention provides a method for the treatment, prevention and control of a disease, said method comprising administering to a subject a composition according to the invention. In one embodiment of the present invention, the disease is a central nervous system related disease.

In one embodiment, the present invention further provides a method for the treatment, prevention and control of stroke, said method comprising administering to a subject an effective amount of one or more molecules according to the invention.

In one embodiment, the present invention further provides a method for the treatment, prevention and control of Parkinson's disease., said method comprising administering to a subject an effective amount of one or more molecules according to the invention.

In one embodiment, the present invention further provides a method for the treatment, prevention and control of Alzheimer's disease., said method comprising administering to a subject an effective amount of one or more molecules according to the invention.

In one embodiment, the present invention further provides a method for the treatment, prevention and control of AIDS dementia, said method comprising administering to a subject an effective amount of one or more molecules according to the invention.

In one embodiment, the present invention further provides a method for the treatment, prevention and control of Huntington's disease, said method comprising administering to a subject an effective amount of one or more molecules according to the invention.

In one embodiment, the present invention further provides a method for the treatment, prevention and control of amyotrophic lateral sclerosis (ALS)., said method comprising administering to a subject an effective amount of one or more molecules according to the invention.

In one embodiment, the present invention further provides a method for the treatment, prevention and control of head trauma., said method comprising administering to a subject an effective amount of one or more molecules according to the invention.

In one embodiment, the present invention further provides a method for the treatment, prevention and control of neurodegenerative or acute brain insults where iNOS and or inflammatory processes involve.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control.

This invention is further illustrated in the Experimental Details section, which follows. This section is set forth to aid in an understanding of the invention but is not intended to, and should not be construed to, limit in any way the invention as set forth in the claims that follow thereafter.

### EXAMPLES:

### Experimental Procedures

### Hybridization Experiments.

The DNA sequences used in the hybridization experiments were purchased from (Sigma; USA). A PNA sequence was purchased from Bio-Synthesis Inc. (Lewisville, USA). All hybrid samples reported were first incubated at 90° C. for 5 minutes, then slowly cooled at room temperature. The Duplex concentration of the various samples was 5 μM. All hybridization experiments were carried out in a 10 mM phosphate buffer, 100 mM NaCl, 0.1 mM EDTA, pH=7. Temperature was monitored by an internal thermocouple, at an accuracy of ±0.3° C. Melting curves were recorded by heating the samples (1° C./min) with a 2-min. hold time by UV signal at 260 nm. Melting temperatures were taken as the maximum of the first derivative of the melting curves. UV absorbance measurements were performed on a Varian Cart 5E UV-VIS-NIR spectrophotometer equipped with a Cary temperature controller, in 1-cm quartz cells.

### Sequences

Sense 5′-3′: GAA AGA GGA AAA GG
Antisenses PNA: CH(K)6HC-O-5′CTTTCTCCTTTTCC3′-O-
HAIYPRH
ODN: CTTTCTCCTTTTCC

| | |
|---|---|
| Sequence | Sense |

| | |
|---|---|
| Oligodeoxynucleotide | 46.96 |
| Calculated PNA | 40 |
| hybridization Tm | |
| Modified PNA | 39.00 |
| experimental value | |

The modifications on the PNA chain although improving bioavailability can diminish activity (hybridization). To examine the effect of modifications on activity properties an experiment was conducted to examine the hybridization properties (determined by changes in temperature of melting or Tm) KBP-10 sequence. Tm value (39.5° C.) was almost the same as predicted calculated value based on sequence (40° C.).

### EXAMPLE 1

### Hybridization Properties of CH(K)6HC-O-5′CTTTCTCCTTTTCC3′-O-HAIYPRH

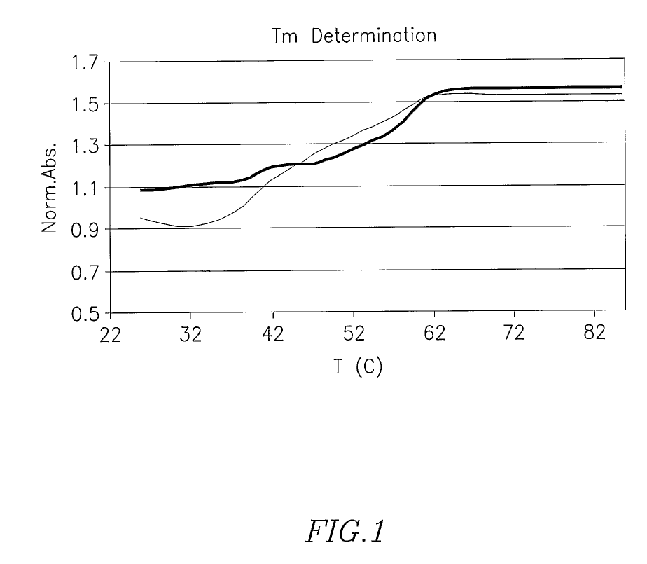

Measurement of Tm revealed that the peptide modification did not affect the hybridization properties of 14 mer homopurine sequence PNA sequence (modified PNA vs. ODN red and black lines respectively-see FIG. 1.).

### EXAMPLE 2

### Uptake of Fluorescence Labeled Unmodified PNA or KBP's Modified PNA (1 μM) to a BBB

In vivo uptake of PNA-Peptide conjugates into mouse brains by in vivo confocal microscopy was use to measure entry of FITC-labeled KBP's modified PNA or radioactive labeled PNAs into mouse brain 1 hour following 15 mg/kg. Data have shown that labeled KBP's modified PNA the uptake of labeled KBP's modified PNA was much greater than the uptake of unmodified PNA.

### EXAMPLE 3

### Multiple Sclerosis Model

Mice (female, Cpb 57B1/6; Harlan) were inoculated with ecephalitogenic peptide (MOG 35-55; synthesized at the Hebrew university) emulsified with CFA and pertussis toxin (Sigma) and in the same time were treated with CH(K)6HC-O-5′CTTTCTCCTTTTCC3′-O-HAIYPRH (KBP-11; 10 mg/kg; n=3) or with vehicle solution (control group; n=4) for 10 consecutive days, starting a day after the inoculation of MOG. Mice were examined for neurological signs of disease as indicated in table II:

| Score | Signs |
|---|---|
| 0 | Normal behavior |
| 1 | Distal limp tail |
| 1.5 | Complete limp tail |
| 2 | Righting reflex |
| 3 | Ataxia |
| 4 | Early paralysis |
| 5 | Full paralysis |
| 6 | Moribund/death |

At the end the experiment (day 30) animals were scarified and brain tissue were send to histological (H&E analysis) and immunohistochemical (iNOS expression) analysis.

| | | Mean disease | |
|---|---|---|---|
| | | duration | Mean Score |
| Group | Incidence | (days) | (Disease burden) |
| PNA | 1/3 | 6 | 0.4 ± 0.16* |
| Control | 4/4 | 11.5 | 1.22 ± 0.12 |

### Immunohistochemistry:

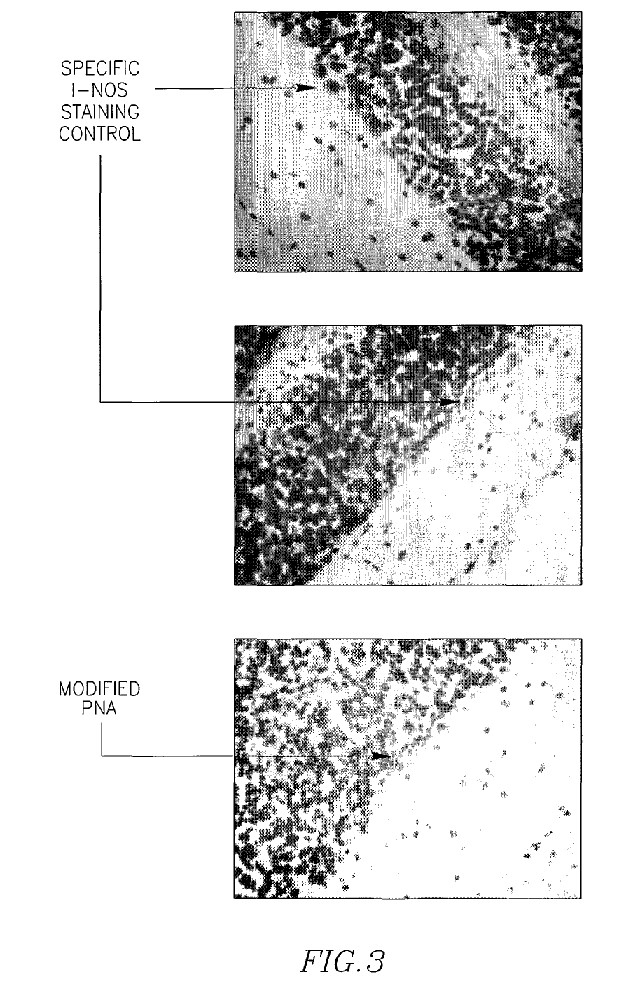

After perfusion with 4% paraformaldehyde, the brains were postfixed for 12 h, cryoprotected in 20% sucrose in saline for 48 h, and frozen in liquid nitrogen-coated isopentane. Coronal sections (12 μm thick) were reacted for 48 h with a rabbit polyclonal iNOS Ab (Santa Cruz Biotechnology;diluted 1:500). After incubation with biotinylated secondary Ab (anti-rabbit IgG, Vector Elite kit, Vector laboratories) and avidin-biotin complex (Vectastatin Elite Kit, Vector Laboratories), the avidin biotin complex was visualized by H2O2 and diaminobenzidine. As is shown in FIG. 3, which shows immunohistochemical staining of iNOS protein in cortical sample of conjugated-PNA treated or control non-treated animals, the conjugated-PNA enabled detection of iNOS.

What we claim is:

1. A molecule represented by any one of the formulas I-IV:
I. [(L)ₜ-(N)ᵣ-(H)_{q}-(P)ₛ]ₓ
II. [(L)ₜ-(H)_{q}-(N)ᵣ-(P)ₛ]ₓ
III. [(P)ₛ-(N)ᵣ-(H)_{q}-(L)ₜ]ₓ
IV. [(P)ₛ-(H)_{q}-(N)ᵣ-(L)ₜ]ₓ
wherein
N is a nucleic acid sequence targeting iNOS, L is a peptide ligand which binds to a specific receptor, P is a positively charge moiety; and H is an hydrophobic moiety wherein r is an integer of 1-25, t is an integer of 1-25, s is an integer of 1-25, q is an integer of 0-20, and x is an integer of 1-20.

2. The molecule according to claim 1, wherein said nucleic acid sequence is a mRNA, a cDNA, a DNA, a DNA analog, a polyamide nucleic acid (PNA), a PNA morpholino, an aminoethylprolyl (aep) PNA, a pyrrolidinyl PNA, an oligonucleotide, an oligonucleotide analog, a ribozyme or an RNAi.

3. The molecule according to claim 1, wherein said nucleic acid sequence is a PNA targeting iNOS, wherein said PNA is DNA, cDNA, RNA or mRNA.

4. The molecule according to claim 1, wherein said nucleic acid sequence is an antisense, an antigene or a decoy function targeting iNOS DNA, cDNA, RNA or mRNA.

5. The molecule according to claim 1, wherein said nucleic acid sequence is neutral or negatively charged.

6. The molecule according to claim 1, wherein said peptide ligand binds a receptor to transferrin, insulin, insulin growth factor, Insulin growth factor or leptin.

7. The molecule according to claim 6, wherein said insulin growth factor is insulin growth factor-I or insulin growth factor-II.

8. The molecule according to claim 1, wherein said peptide ligand has amino acid sequence, which is HAIYPRH or THRPPMWSPVWP.

9. The molecule according to claim 1, wherein said hydrophobic moiety is a nucleic acid.

10. The molecule according to claim 1, wherein said hydrophobic moiety is a hydrophobic peptide, lipid acid, lipid molecules, octanol, cholesterol, hydrophobic peptide protecting group, adamantine, pyrene, eicosenoic acid, C_{(6-16) glyceride lipid, phenoxazine, DMT group, cholenic acid, lithocholic acid, myristic acid, palmitic acid, heptadecyl group, hexadecylglycerol, geranyloxyhexyl group, hexadecylamine, dihydrotestosterone,} 1-pyrene butyric acid, alkanoic acid, alkanol or any derivatives thereof.

11. The molecule according to claim 10, wherein said alkanoic acid is represented by the structure R—(CH₂)n−COOH, wherein n=1-20 and R is a linear or branched alkyl.

12. The molecule according to claim 10, wherein said alkanol is represented by the structure R—(CH₂)n—OH, wherein n=1-20 and R is a linear or branched alkyl.

13. The molecule according to claim 10, wherein said lipid acid is undecanoic acid and/or docosahexanenonic acid.

14. The molecule according to claim 10, wherein said hydrophobic peptide protecting group is Fmoc or Tboc.

15. The molecule according to claim 1, wherein said positively charge moiety is a nucleic acid.

16. The molecule according to claim 1, wherein said positively charge moiety is positively charge peptide, peptidomimetic, polycations, histidine, imidazole group, 2-O-aminopropyl, 2-O-dimethylaminopropyl, 2-O-imidazolyl-ethyl, 2-O-aminoethylamino-oxyethyl, 2-dimethylaminoethyl-oxyethyl or any derivative thereof.

17. The molecule according to claim 1, wherein said positively charge moiety comprises at least one group of arginine, polyamine and/or guanidine.

18. The molecule according to claim 17, wherein said polyamine is spermine, spermidine or putricine.

19. The molecule according to claim 1, wherein said peptide ligand, said nucleic acid sequence targeting iNOS, said hydrophobic moiety, and said positively charge moiety are linked to each other directly via peptide bonds.

20. The molecule according to claim 1, further comprising a linker moiety linking between said peptide ligand, said hydrophobic moiety, said nucleic acid sequence targeting iNOS and said positively charge moiety.

21. The molecule according to claim 20, wherein said linker moiety is polyethylene glycol, disulfide, amide, amine, oxyamine, oxyimine, morpholine, thioether, thiourea sulfonamide, ether, ester, carbonate, carbamate, avidin, strepavidin, biotin, praline, lysine, cysteine, guanidine or any combination thereof.

22. The molecule according to claim 21, wherein the molecular weight of said polyethylene glycol is in the range of 2000-40,000.

23. A molecule represented by any one of the formulas V-VIII:
V. [(L)ₜ-(PNA)ᵣ-(H)_{q}-(P)ₛ]ₓ
VI. [(L)ₜ-(H)_{q}-(PNA)ᵣ-(P)ₛ]ₓ
VII. [(P)ₛ-(PNA)ᵣ-(H)_{q}-(L)ₜ]ₓ
VIII. [(P)ₛ-(H)_{q}-(PNA)ᵣ-(L)ₜ]ₓ
wherein the PNA is PNA sequence targeting iNOS DNA, cDNA, RNA or mRNA and is 1-100 bases, L is a peptide ligand which binds to a specific receptor and P is a positively charge moiety; and wherein r is an integer of 1-25, t is an integer of 1-25 s is an integer of 0-25, q is an integer of 0-20 and x is an integer of 1-20.

24. The molecule according to claim 23, wherein said PNA sequence targeting iNOS DNA, cDNA, RNA or mRNA is an antisense, an antigene or a decoy function.

25. The molecule according to claim 23, wherein said PNA sequence targeting iNOS DNA, cDNA, RNA or mRNA is neutral or negatively charged.

26. The molecule according to claim 23, wherein said peptide ligand binds a receptor to transferring, insulin, insulin growth factor, Insulin growth factor or leptin.

27. The molecule according to claim 26, wherein said insulin growth factor is insulin growth factor-I or insulin growth factor-II.

28. The molecule according to claim 23, wherein said peptide ligand has amino acid sequence, which is HAIYPRH or THRPPMWSPVWP.

29. The molecule according to claim 23, wherein said hydrophobic moiety is a nucleic acid.

30. The molecule according to claim 23, wherein said hydrophobic moiety is hydrophobic peptide, lipid acid, lipid molecules, octanol, cholesterol, hydrophobic peptide protecting group, adamantine, pyrene, eicosenoic acid, C(₆-16) glyceride lipid, phenoxazine, DMT group, cholenic acid, lithocholic acid, myristic acid, palmitic acid, heptadecyl group, hexadecylglycerol, geranyloxyhexyl group, hexadecylamine, dihydrotestosterone, 1-pyrene butyric acid, alkanoic acid, alkanol or any derivatives thereof.

31. The molecule according to claim 30, wherein said alkanoic acid is represented by the structure R—(CH₂)n—COOH, wherein n=1-20 and R is a linear or branched alkyl.

32. The molecule according to claim 30, wherein said alkanol is represented by the structure R—(CH₂)n—OH, wherein n=1-20 and R is a linear or branched alkyl.

33. The molecule according to claim 30, wherein said lipid acid is undecanoic acid and/or docosahexanenonic acid.

34. The molecule according to claim 30, wherein said hydrophobic peptide protecting group is Fmoc or Tboc.

35. The molecule according to claim 23, wherein said positively charge moiety is a nucleic acid targeting iNOS DNA, cDNA, RNA or mRNA.

36. The molecule according to claim 23, wherein said positively charge moiety is positively charge peptide, peptidomimetic, polycations, histidine, imidazole group, 2-O-aminopropyl, 2-O-dimethylaminopropyl, 2-O-imidazolyl-ethyl, 2-O-aminoethylamino-oxyethyl, 2-dimethylaminoethyl-oxyethyl or any derivative thereof.

37. The molecule according to claim 23, wherein said positively charge moiety comprises at least one group of arginine, polyamin and/or guanidine.

38. The molecule according to claim 37, wherein said polyamine is spermine, spermidine or putricine.

39. The molecule according to claim 23, wherein said peptide ligand, said PNA sequence targeting iNOS DNA, cDNA, RNA or mRNA, said hydrophobic moiety and said positively charge moiety are linked to each other directly via peptide bonds.

40. The molecule according to claim 23, further comprising a linker moiety linking between said peptide ligand, said hydrophobic moiety, said PNA sequence and said positively charge moiety.

41. The molecule according to claim 40, wherein said linker moiety is polyethylene glycol, disulfide, amide, amine, oxyamine, oxyimine, morpholine, thioether, thiourea sulfonamide, ether, ester, carbonate, carbamate, avidin, strepavidin, biotin, praline, lysine, cysteine, guanidine or any combination thereof.

42. The molecule according to claim 4 1, wherein the molecular weight of said polyethylene glycol is in the range of 2000-40,000.

43. A composition is comprising as an active ingredient an effective amount of one or more molecules according to claim 1, together with one or more pharmaceutically acceptable excipients or adjuvants.

44. A composition comprising as an active ingredient an effective amount of one or more molecules according to claim 23, together with one or more pharmaceutically acceptable excipients or adjuvants.

45. The composition according to claim 43, formulated for oral or parenteral administration.

46. The composition according to claim 44, formulated for oral or parenteral administration.

47. The composition according to claim 43, formulated as uncoated tablets, coated tablets, pills, capsules, powder or suspension.

48. The composition according to claim 44, formulated as uncoated tablets, coated tablets, pills, capsules, powder or suspension.

49. The composition according to claim 43, formulated for intravenous administration.

50. The composition according to claim 44, formulated for intravenous administration.

51. The composition according to claim 43, formulated for intranasal administration.

52. The composition according to claim 44, formulated for intranasal administration.

53. The composition according to claim 43, formulated for administration via aerosols.

54. The composition according to claim 44, formulated for administration via aerosols.

55. The composition according to claim 43, formulated for transdermal administration.

56. The composition according to claim 44, formulated for transdermal administration.

57. The composition according to claim 43, formulated in an ointment, cream or gel form.

58. The composition according to claim 44, formulated in an ointment, cream or gel form.

59. A method for the synthesis of a molecule according to claim 23.

60. A method for delivering a nucleic acid sequence targeting iNOS DNA, cDNA, RNA or mRNA across a cellular membrane comprising the step of applying to a cell an effective amount of one or more molecules according to claim 1.

61. The method according to claim 60, wherein said cell is an endothelial cell.

62. The method according to claim 60, wherein said cell is a neuronal cell.

63. The method according to claim 60, wherein said cell is glial cell.

64. A method for delivering a PNA sequence targeting iNOS DNA, cDNA, RNA or mRNA across a cellular membrane comprising the step of applying to a cell an effective amount of one or more molecules according to claim 23.

65. The method according to claim 64, wherein said cell is an endothelial cell.

66. The method according to claim 64, wherein said cell is a neuronal cell.

67. The method according to claim 64, wherein said cell is glial cell.

68. The method according to claim 64, wherein said cell is oligodendrocyte cell.

69. A method for intracellular targeting of a nucleic acid sequence targeting iNOS DNA, cDNA, RNA or mRNA to an intracellular organelle comprising the step of applying to a cell an effective amount of one or more molecules according to claim 1.

70. A method for intracellular targeting of a PNA sequence targeting iNOS DNA, cDNA, RNA or mRNA to an intracellular organelle comprising the step of applying to a cell an effective amount of one or more molecules according to claim 23.

71. A method for intracellular targeting of a nucleic acid sequence targeting iNOS DNA, cDNA, RNA or mRNA to an intracellular organelle comprising the step of applying to a cell an effective amount of one or more molecules according to claim 1, wherein said molecule crosses the nuclear membrane.

72. A method for intracellular targeting of a PNA sequence targeting iNOS DNA, cDNA, RNA or mRNA to an intracellular organelle comprising the step of applying to a cell an effective amount of one or more molecules according to claim 23, wherein said molecule crosses the nuclear membrane.

73. A method for delivering a nucleic acid sequence targeting iNOS DNA, cDNA, RNA or mRNA to the brain across the blood brain barrier, said method comprising the step of administering to a subject an effective amount of one or more molecules according to claim 1.

74. A method for delivering a PNA sequence targeting iNOS DNA, cDNA, RNA or mRNA to the brain across the blood brain barrier, said method comprising the step of administering to a subject an effective amount of one or more molecules according to claim 23.

75. A method for delivering a nucleic acid sequence targeting iNOS DNA, cDNA, RNA or mRNA to the brain across the blood brain barrier, said method comprising the step of administering to a subject a composition according to claim 43.

76. A method for delivering a PNA sequence targeting iNOS DNA, cDNA, RNA or mRNA to the brain across the blood brain barrier, said method comprising the step of administering to a subject a composition according to claim 44.

77. A method for delivering a PNA sequence targeting iNOS DNA, cDNA, RNA or mRNA to the spinal cord, said method comprising administering to a subject an effective amount of one or more molecules according to claim 1.

78. A method for modulating iNOS gene expression, said method comprising administering to a subject an effective amount of one or more molecules according to claim 23.

79. A method for modulating iNOS gene expression, said method comprising administering to a subject one a composition according to claim 43.

80. A method for modulating iNOS gene expression, said method comprising administering to a subject one a composition according to claim 44.

81. A method for the treatment, prevention and control of a disease as a result of inhibition of iNOS translation, said method comprising administering to a subject an effective amount of one or more molecules according to claim 1.

82. The method of claim 81, wherein said disease is multiple sclerosis.
